(19) United States
(12) Patent Application Publication
(10) Pub. No.: US 2025/0111499 A1
(43) Pub. Date: Apr. 3, 2025

(54) SYSTEMS AND METHODS FOR HEALTH CARE SCREENING USING A HEAD-MOUNTED DISPLAY

(71) Applicant: Adeia Guides Inc., San Jose, CA (US)

(72) Inventors: Kamal J. Koshy, Aurora, CO (US); Reda Harb, Tampa, FL (US); Dhananjay Lal, Englewood, CO (US)

(21) Appl. No.: 18/374,099

(22) Filed: Sep. 28, 2023

(51) Int. Cl. G06T 7/00; A61B 3/00; A61B 3/11; A61B 5/00; G16H 40/67; H04N 23/60; H04N 23/661

(52) U.S. Cl. CPC G06T 7/0012; A61B 3/0091; A61B 3/112; A61B 5/0077; A61B 5/443; A61B 5/6803; A61B 5/7445; G16H 40/67; H04N 23/64; H04N 23/661; G06T 2207/30041; G06T 2207/30201

(57) ABSTRACT

Systems and methods are provided for performing preliminary health care screening using a head-mounted display to provide preliminary health care screening for some conditions and diseases that manifest symptoms in the eyes or in the ocular facial region around the eyes through interaction with an analysis agent. The head-mounted display is worn by a wearer and receives imaging instructions from an analysis agent. The head-mounted display generates screening images of the eyes and the ocular facial region of the wearer based upon the imaging instructions. The screening images are transmitted to the analysis agent, and in response, the head-mounted display receives further imaging instructions from the analysis agent. The further imaging instructions may include parameters for generating more images, a suggestion to contact a health care professional, and/or if pre-approved, notice that a referral has already been made to a health care professional.

### BACKGROUND

This disclosure is generally directed to systems and methods for health care screening using a head-mounted display. In particular, systems and methods are provided herein that generate images of the eyes, and the ocular facial region around the eyes, of a wearer of a head-mounted display, and those images may be analyzed for purposes of providing health care screening for early identification of symptoms of certain conditions and diseases.

### SUMMARY

Head-mounted displays (HMDs) have been gaining in popularity as they are used for virtual reality (such as in gaming and education) and augmented reality (such as in medicine and engineering), among other uses. The primary purpose of HMDs is to present entertainment and/or information graphically to a wearer, and in order to fulfill this purpose, many types of HMDs are essentially head-mounted computing systems, including wearer-facing cameras to measure the wearer's eye movement. Some HMDs include both a visible spectrum camera and an infrared spectrum camera to track eye movement. Having cameras regularly directed at the eyes of the wearer of a head-mounted display (HMD) has previously been unappreciated as a preliminary screening tool for potential health issues. Such potential health issues include those that manifest visibly detectable symptoms in the eyes and the tissues surrounding the eyes. Such potential health issues of this type include xanthelasma (high cholesterol), strabismus, Lyme disease, and thyroid eye disease. Such conditions are at present easily diagnosed through a visit with a qualified medical professional. However, most people don't tend to notice the symptoms until they become highly visually apparent or are accompanied by other symptoms of the underlying disease. And, because some of the symptoms are not seen as a priority until they begin to interfere with everyday life, people with the symptoms tend ignore them. In addition, some people don't make regular visits to medical professionals because such visits can be associated with significant barriers and costs, including transportation, taking time off work, the cost of health care, and the like. An HMD that monitors the wearer's eyes and ocular facial region for early detection and provides either feedback to the wearer or notification to the wearer's chosen health care professional, can help provide early detection and treatment.

There exists a need, therefore, for HMDs for use in generating images of the eyes and ocular facial region of a wearer so that those images may be analyzed as a preliminary screening for early indicators of health issues detectable through such images. Accordingly, the systems and methods disclosed herein provide for an HMD that includes an imaging system for generating images of a wearer's eyes and the ocular facial region around the eyes in accordance with imaging parameters so that preliminary health care screening can be performed for purposes of early detection of symptoms for certain conditions and diseases.

As discussed in greater detail below, the systems and methods presented herein enable an HMD to provide preliminary health care screening for some diseases and/or conditions that manifest visually identifiable symptoms in the eyes or in the ocular facial region around the eyes through interaction with an analysis agent. The head-mounted display includes a display screen for the wearer; an imaging subsystem, e.g., one or more cameras to generate images of the wearer's ocular facial region (using the display screen or other included lighting source for illumination); storage for saving, managing, and processing generated images; and a communications interface (that uses a protocol allowing two or more computing devices to communicate with each other (e.g., WiFi, Bluetooth, USB, Ethernet, serial, and the like) to communicate with the analysis agent. Such systems and methods are configured to interact with the analysis agent, which uses deep machine learning to analyze images of the wearer's eyes and ocular facial region. The deep machine learning aspect of the analysis agent may be initially trained using baseline training images generated by medial institutions and their patients. Following initial training, the head-mounted display obtains context training images of the wearer while the head-mounted display is worn. The purpose for the context training images is to contextually train the analysis agent using images obtained in the environment of the worn head-mounted display, as this environment is unlike the environment of medical offices. This retraining assists the analysis agent in determining how the early indicators of health issues might appear under the lighting conditions created when the head-mounted display is worn. Moreover, retraining for each individual wearer assists the analysis agent in better accounting for the differences in fit of the head-mounted display for different wearers, variations in skin tone between wearers, variations in facial features between wearers, and the like.

In some embodiments, the systems and methods are presented whereby the head-mounted display is worn and receives imaging instructions, comprising imaging parameters, from the analysis agent. The head-mounted display comprises an imaging subsystem and a display screen, which are used to generate screening images, based upon the imaging parameters, of the ocular facial region of the wearer. The head-mounted display transmits the screening images to the analysis agent via a communications interface, and following analysis by the analysis agent, the head-mounted display receives further imaging instructions from the analysis agent. In some embodiments, the further imaging instructions may instruct the head-mounted display to generate additional screening images for further analysis. In addition, the further imaging instructions may indicate an approximate time frame for generating additional screening images. In some embodiments, this cycle of generating screening images, transmitting the screening images for analysis, and receiving further imaging instructions may be repeated indefinitely to continue conducting preliminary health screenings for the wearer. In certain embodiments, the head-mounted display may generate training images, transmit the training images to the analysis agent, which may use the training images for purposes of training a neural network model, and then receive imaging instructions from the analysis agent to begin the health screening process.

### BRIEF DESCRIPTION OF THE DRAWINGS

The above and other objects and advantages of the disclosure may be apparent upon consideration of the following detailed description, taken in conjunction with the accompanying drawings, in which:

FIG. 1 schematically illustrates an example environment in which a head-mounted display is configured to perform health care screening of the wearer through communication with and analysis by an analysis agent;

FIG. 2 shows a display screen face of a head-mounted display;

FIG. 3 shows a flowchart of illustrative steps involved in a head-mounted display performing health care screening of the wearer through communication with and analysis by an analysis agent;

FIG. 4 shows a flowchart of illustrative steps involved in supporting a head-mounted display performing health care screening of the wearer;

FIG. 5 shows a flowchart of illustrative steps involved in pre-training an analysis agent for health care screening of a wearer;

FIG. 6 illustrates an example of a first health condition in which a head-mounted display may be used to perform a health-care screening for a wearer;

FIG. 7 illustrates an example of a second health condition in which a head-mounted display may be used to perform a health-care screening for a wearer;

FIG. 8 illustrates an example of a third health condition in which a head-mounted display may be used to perform a health-care screening for a wearer;

FIG. 9 illustrates an example of a fourth health condition in which a head-mounted display may be used to perform a health-care screening for the wearer; and

FIG. 10 is a block diagram of a head-mounted display configured to perform health-care screening and incorporated into a communication system which includes a network and analysis agent.

### DETAILED DESCRIPTION

As referred to herein, the term “content” should be understood to mean an electronically consumable asset accessed using any suitable electronic platform, such as broadcast television, pay-per-view, on-demand (as in video-on-demand (VOD) systems), network-accessible media (e.g., streaming media, downloadable media, Webcasts, etc.), video clips, audio, haptic feedback, information about media, images, animations, documents, playlists, websites and webpages, articles, books, electronic books, blogs, chat sessions, social media, software applications, games, virtual reality media, augmented reality media, and/or any other media or multimedia and/or any combination thereof. Extended reality (XR) content, which is a particular type of content, refers to augmented reality (AR) content, virtual reality (VR) content, hybrid or mixed reality (MR) content, and/or other digital content combined therewith to mirror physical-world objects, including interactions with such content.

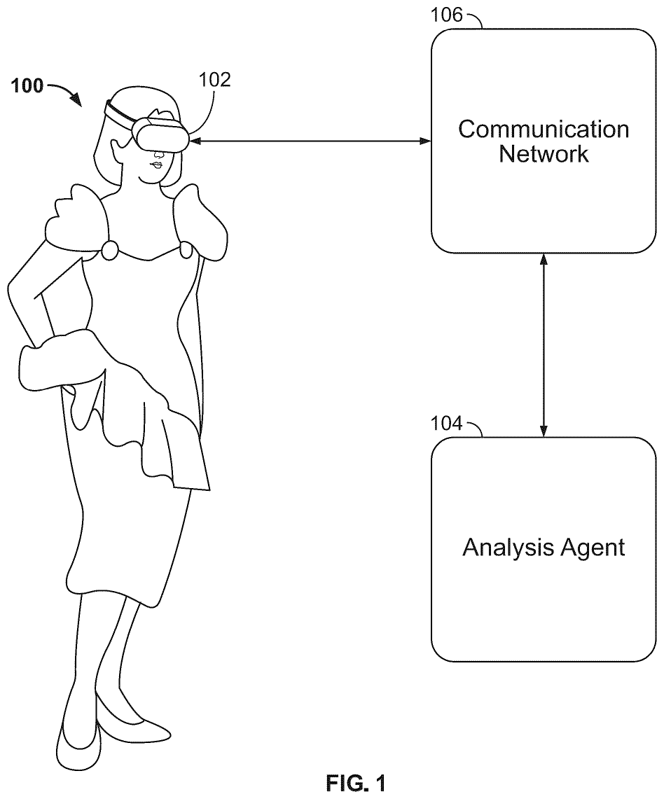

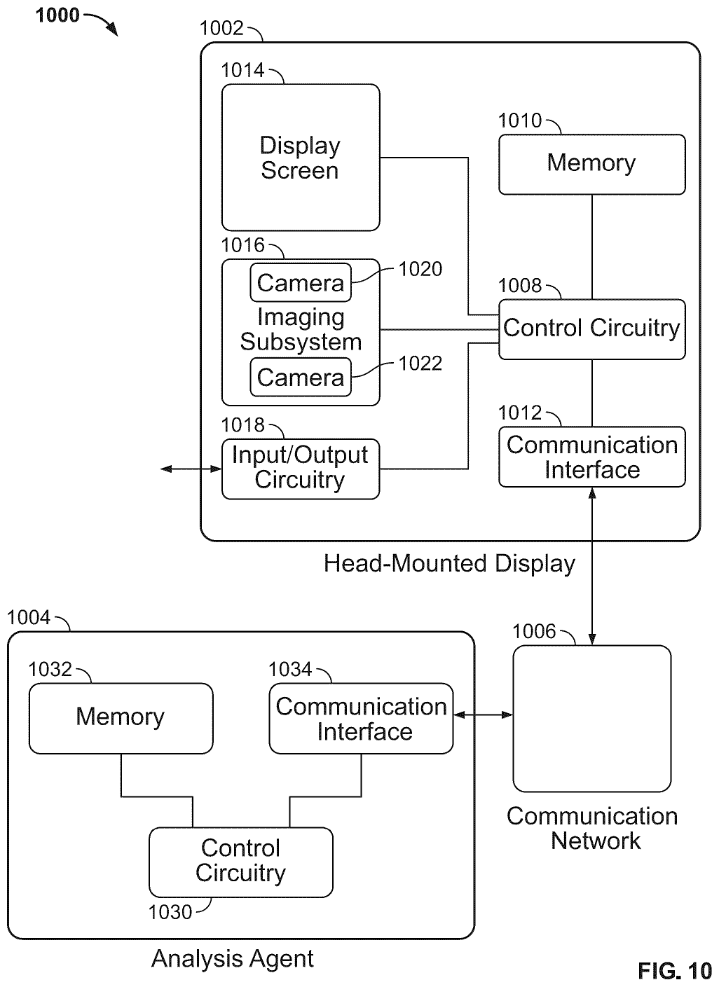

Turning in detail to the drawings, FIG. 1 shows a first example of an environment in which a head-mounted display (HMD) 102 is configured to perform health care screening of the wearer 100. The head-mounted display 102 communicates with an analysis agent 104 via a network 106 to communicate screening images of the wearer 100 to the analysis agent 104 for analysis. In some embodiments, the HMD 102 may be any wearable device used for viewing content and/or for interacting with extended reality (XR) content. The HMD 102 includes control circuitry, an imaging subsystem, a display screen, and communications circuitry (see FIG. 10 and accompanying description for details concerning these components). Briefly, the control circuitry controls the operational functionality of the various components of the HMD 102; the imaging subsystem aids in generating images of the wearer's eyes and ocular facial region; and the display screen both displays content to the wearer 100, and as needed, serves as a highly versatile light source when images are being generated. The communications interface enables the HMD to transmit and receive data, content requests, and the like (including XR content, multimedia content, and images generated by the HMD 102) to and from other devices, and the communications interface may be configured for wired communications, wireless communications, or both. As is described in further detail below, the HMD 102 generates screening images of the wearer's eyes and ocular facial region and transmits the screening images to the analysis agent 104 for analysis. The HMD 102 may perform additional process steps, such as generating additional screening images, based upon instructions received from the analysis agent 104 following analysis of the screening images.

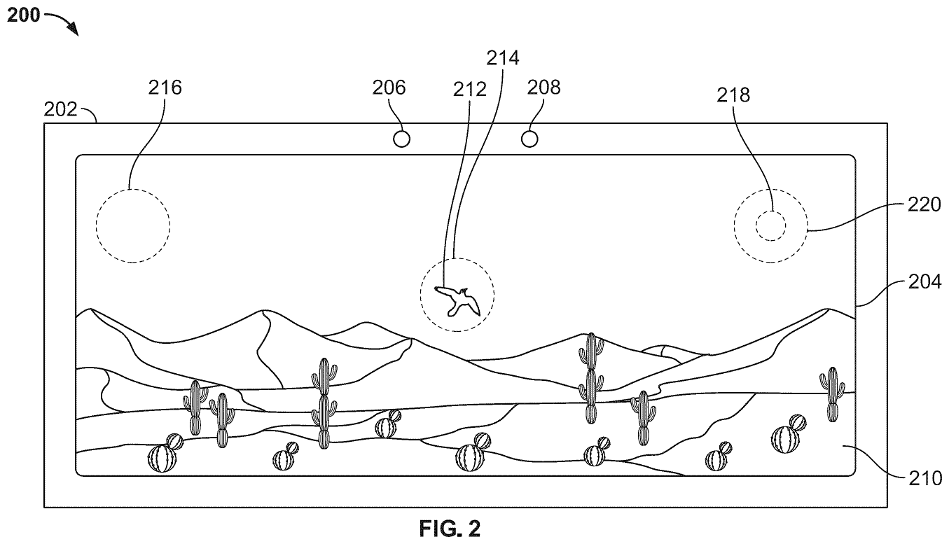

An embodiment of the display face 200 of the HMD 102 is shown in FIG. 2. When the HMD 102 is worn, the display face 200 is positioned in front of the wearer's eyes. In some embodiments, the distance between the wearer's eyes and the display face 200 may be within the industry standard range of approximately 1 cm to 2 cm. In some embodiments, depending upon other applications for which the HMD 102 is used, the distance between the wearer's eyes and the display may be outside of this industry standard.

The display face 200 includes a bezel 202 bordering a display screen 204, and two cameras 206, 208 are located in the bezel 202. Each camera 206, 208 is part of the imaging subsystem of the HMD 102, and each is positioned so that an image of each of the wearer's eyes and ocular facial region may be generated. In some embodiments, the HMD 102 may include a single camera positioned to image both eyes. In some embodiments, the cameras 206, 208 may also include the capabilities to generate images in the infrared spectrum. In some embodiments, the HMD 102 may include additional cameras configured to generate images in the infrared spectrum.

As shown, the display screen 204 is a single contiguous screen. In some embodiments, the display screen 204 may be formed by a screen having two distinct sections, with each section being placed so that each is positioned in front of one of the wearer's eyes when the HMD is worn. During use, the display screen 204 is controlled to show a different image to each of the wearer's eyes, corresponding to a left eye image and a right eye image, and in doing so, the wearer perceives the screening image 210 as a unified visual field that provides the wearer with a perception of depth. The screening image 210 illustrated on the display screen 204 in FIG. 2 is shown as a desert landscape, and it is shown as perceived by the wearer, i.e., a unified visual field, as opposed to the split screen images that would be shown to the wearer to create the unified visual field. During use, the screening image 210 perceived by the wearer may be incorporated as part of any type of visual media content displayed as a scene, an image, or a video, and that visual media may be pre-composed (e.g., a movie or other pre-recorded video, images, and the like), created in the moment (e.g., video from a video game being played by the wearer), or a mixture of both. In certain embodiments, the screening image 210 presented may be multimedia and include audio along with the visual media. In some embodiments, the screening image 210 may be media content that is originally created for purposes of the health screening discussed herein. In some other embodiments, the screening image 210 may have a primary purpose other than for the health screening (e.g., a game, a movie, a presentation), with the screening image 210 being adapted for use with the health screening discussed herein.

Several focus zones 214, 216, 218, 220 may be defined within the screening image 210 perceived by the wearer. These focus zones 214, 216, 218, 220 represent positions and focal points to which the wearer's eyes are to be directed while images are generated for a health care screening. Each focus zone 214, 216, 218, 220 is shown as a broken line circle for purposes of illustration only. As shown, the screening image 210 includes an object image 212, illustrated as a flying bird, within focus zone 214. In some embodiments, the size of each focus zone 214, 216, 218, 220 is used to define the wearer's perceived depth of an object image 212 having a position determined by one of the focus zones 214, 216, 218, 220. In such embodiments, the size of each focus zone 214, 216, 218, 220 defines both the position and size of an object image. For example, focus zones 218, 220 are shown as concurrent circles, with focus zone 218 being represented by a smaller circle than focus zone 220. Thus, to the wearer, an object image having a position determined by focus zone 218 will appear smaller and at a greater depth of field within the screening image 210 as compared to an object image having a position determined by focus zone 220.

In some embodiments, the screening image 210 may include more or fewer focus zones, and the focus zones 214, 216, 218, 220 may be placed at any location within the screening image 210 in support of some aspect of the health care screening process. In some embodiments, an object image may remain within one of the focus zones 214, 216, 218, 220 during the health care screening process. In some embodiments, the multiple focus zones 214, 216, 218, 220 may be defined and used in combination to support the health care screening process. In such embodiments, an object image may be moved between any two or more of the focus zones 214, 216, 218, 220 to encourage the wearer's eyes to track the movement as part of the health care screening process.

In some embodiments, audible instructions may be used in combination with defined focus zones to support the health care screening process. For example, an object image may be positioned within one of the focus zones 214, 216, 218, 220, and the audible instructions may instruct the wearer to look at the object image and then blink or close their eyes. Such a sequence may be used to generate images of the skin immediately around the wearer's eyes in support of the health care screening process.

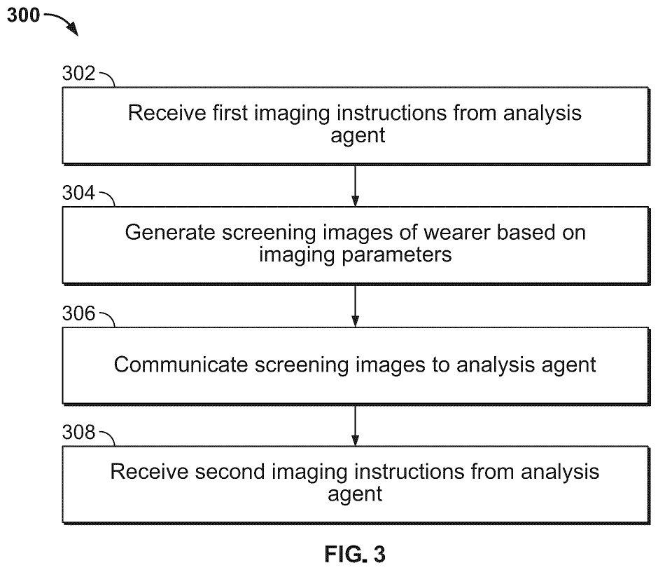

FIG. 3 shows a flowchart illustrating the steps of a process 300 for enabling an HMD to perform health care screening. The process 300 may be implemented on the HMD 102 discussed herein and similar user devices for displaying XR content to a wearer. One or more actions of the process 300 may be incorporated into or combined with one or more actions of any other process or embodiments described herein. At step 302, the HMD receives, via a communications interface, first imaging instructions from an analysis agent. The first imaging instructions include detailed instructions to the HMD for generating, processing, and communicating screening images. As part of the detailed instructions for generating screening images, the first imaging instructions include imaging parameters. At step 304, the HMD generates, using the imaging sub-system and the display screen, screening images of the eyes and ocular facial region of the wearer based on the imaging parameters from the received first imaging instructions. Once the screening images are generated in accordance to the imaging parameters, at step 306 the screening images are transmitted, via the communications interface, to the analysis agent. Then, at step 308, the HMD receives, via the communications interface, second imaging instructions from the analysis agent.

In some embodiments, the process 300 may be implemented as an iterative process. In such embodiments, the second imaging instructions may include second imaging parameters, and the HMD would generate additional screening images based on the second imaging parameters. Those additional screening images would be transmitted to the analysis agent and analyzed, and the HMD would receive yet more imaging instructions from the analysis agent in response. In some embodiments, this iterative process could proceed for any desired amount of time to continue health care screening for the wearer. In some embodiments, subsequent imaging instructions may instruct the HMD to generate screening images while the wearer is using the HMD for other purposes (e.g., enjoying a VR world, gaming, watching a movie, etc.). In such an embodiment, generation of the screening images, and the subsequent analysis of the screening images, would occur without the wearer's active participation in the process. The wearer would become aware that the screening images were generated and analyzed only upon receiving notice about the results of the screening.

The imaging parameters define how the HMD is to generate one or more of the screening images. In some embodiments, the imaging parameters may define or identify a predetermined scene to be displayed on the display screen while one or more of the screening images is generated. The predetermined scene may be a still image, a video, an illumination setting for the display screen, or other visual media. In instances when the predetermined scene is an illumination setting, the display screen may display a blank screen and adjust the settings of the display screen to match the illumination settings. Such illumination settings may be used to establish the color of the display screen, the brightness of the display screen, the warmth of illumination from the display screen, and the like. The imaging parameters may also specify whether an object image should be displayed on the display screen while the HMD generates one or more of the screening images. For example, the imaging parameters may indicate that an object image should be displayed in a defined focus zone (e.g., 214, 216, 218, 220 in FIG. 2), to induce the wearer to look in a predetermined direction, while the screening images are generated. In some embodiments, the imaging parameters may indicate that an object should be moved from one focus zone to another focus zone, to induce the wearer to track the object image with their eyes, while the screening images are generated. In some embodiments, the imaging parameters may specify that a predetermined visual media (i.e., pre-existing video or interactive video) is displayed to the wearer while the screening images are generated because such predetermined visual media is already known to the analysis agent to induce the wearer to look in a predetermined direction and/or to cause the wearer to track their eyes from one focus point to another focus point. In some embodiments, the imaging parameters may define a range or ranges for one or more illumination settings, and these defined ranges may be used for determining when screening images should be generated while the wearer is viewing unknown visual media in the HMD. For example, the wearer may be watching a movie while wearing the HMD, and the imaging parameters may define a range for average brightness and a range for average color of the display screen. At any point during display of the movie to the wearer, if the display screen has an average brightness and average color that fall within the defined ranges, then the HMD generates one or more screening images.

In some embodiments, the imaging instructions may define how many screening images should be generated by the HMD and/or a schedule for generating the screening images. In some embodiments, the imaging instructions may also include additional instructions to the HMD or information relating to the screening analysis. The additional instructions may be directed toward processing, storing, or further communicating the screening images. For example, the additional instructions may direct the HMD to communicate the screening images to a network storage for backup or otherwise storing the screening images for future reference. As another example, the additional instructions may direct the HMD to present the screening images to the wearer along with a brief explanation of the screening analysis and why the wearer should see a medical practitioner. In some embodiments, with the wearer's prior authorization, the additional instructions may direct the HMD to communicate the screening analysis and screening images to a health care system or health care provider. In doing so, the HMD facilitates the wearer seeking a consultation with a medical practitioner. In some embodiments, the information relating to the screening analysis may include the results of the screening analysis itself, notes for a medical practitioner (which can be provided in electronic form directly by the HMD or in by the wearer themselves), and/or information to the wearer about the screening process.

The first imaging instructions may be open-ended or closed-ended. As open-ended instructions, the first imaging instructions may instruct the HMD to continue generating screening images in accordance with the imaging parameters and communicating the generated screening images to the analysis agent until receiving second imaging instructions. As closed-ended instructions, the first imaging instructions may instruct the HMD to generate screening images in accordance with the imaging parameters, communicate the generated screening images to the analysis agent, and then cease generating screening images until receiving second imaging instructions, with second imaging parameters, from the analysis agent.

The process 300 may be integrated into the HMD as a health care screening module (software or hardware based) with application programming interface (API) access to the XR engine and other components integrated (e.g., display screen, eye tracker, imaging subsystem, audio output, communications interface, etc.) as part of the HMD. Through the API access, the health care screening module may utilize components of the HMD to generate the screening images used for the health care screening process and analysis.

During the process 300, the health care screening module communicates with the analysis agent by sending screening images for analysis and receiving imaging instructions. The analysis agent is a neural network model of a type that can be trained to perform image recognition effectively and accurately, such that once trained, the neural network is able to analyze new images to and classify them as having a disease and/or condition or not. In some embodiments, the neural network may be a deep neural network, a convolution neural network, and the like. Initially, the neural network is pre-trained using initial training images showing diseases and/or conditions that a medical practitioner can detect by visual inspection or eye examination. These training images are already classified to identify the disease(s) and/or condition(s) for which the patient was diagnosed. To further the abilities of the neural network to identify diseases and/or conditions, during pre-training the neural network may be trained using sematic segmentation to improve classification of diseases and/or conditions. Through semantic segmentation, pixels may be identified to specify to the model specific parts of the eye in training images along with parts of the eye where symptoms may occur. For example, in some embodiments semantic segmentation may be used to identify the pupil, the iris, the sclera, the rims of the eye lids, the eyelids when the eyelids are open, the eyelids when the eyelids are shut, any region around the eyes in which symptoms of a disease and/or condition may be visible, and any other physical features of the eyes and ocular facial region that may be used or are related to visible symptoms. In some embodiments, the capabilities of the neural network model may be enhanced by building a database of training images of individuals looking in various directions so that the neural network model may be trained on images of the eyes from many different directions. In some embodiments, this contextual training can be effectively achieved with a smaller set of images than used for the pre-training. In some embodiments, historical images of the wearer may be provided for further training of the neural network model. Such historical images are particularly helpful to training when the historical images show the wearer having an existing disease and/or condition that may be diagnosed by visual inspection of the eyes.

In general, the initial training images are taken in the setting of a medical office, and such settings generally have bright lighting. The initial training images, therefore, are taken in a different environment than the environment that is present within the confines of an HMD. Therefore, as discussed further below, in some embodiments the neural network may be further trained with training images generated within the context of the HMD.

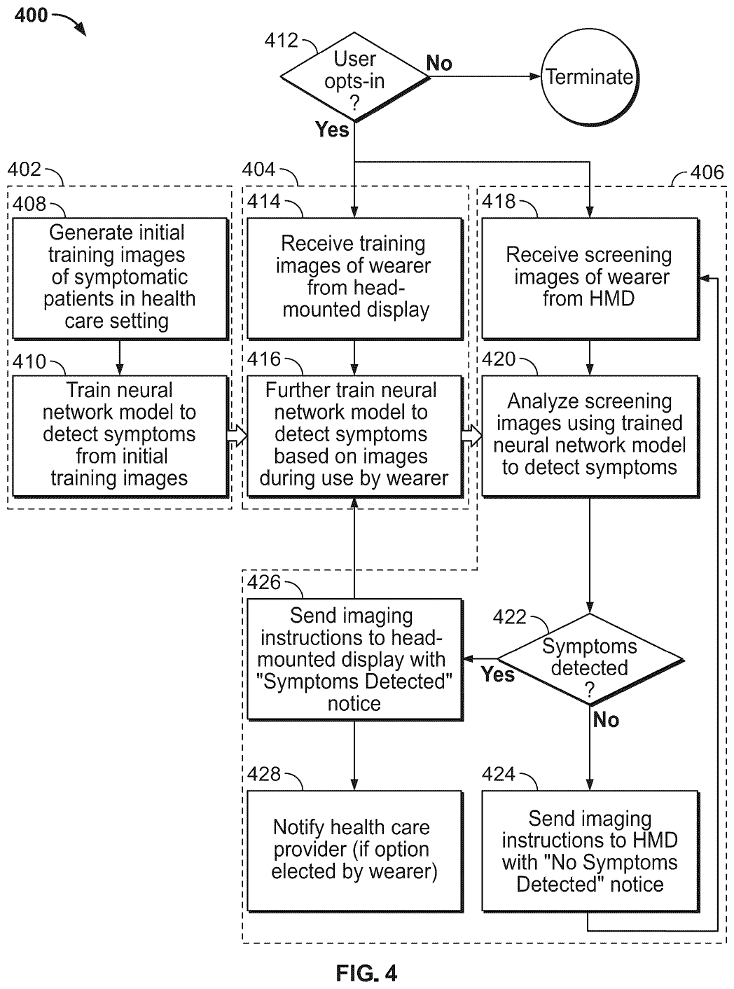

FIG. 4 shows a flowchart illustrating the steps of a process 400 for enabling an analysis agent to interact with an HMD to perform health care screening. The process 400 may be implemented on the analysis agent 104 in support of the health care screening process 300 of the HMD 102. One or more actions of the process 400 may be incorporated into or combined with one or more actions of any other process or embodiments described herein. The process 400 includes a pre-training stage 402, a contextual training stage 404, and a health care screening stage 406. In the pre-training stage 402, at step 408, initial training images of symptomatic patients are generated in a health care setting (e.g., a medical office). At step 410, the neural network model is trained to detect symptoms using the initial training images. The symptoms detected are generally those which would ordinarily be detectible by a medical practitioner through visual inspection or eye examination of a patient. The pre-trained neural network model may then be used as the baseline for the neural network of the analysis agent, and using the neural network model, the analysis agent is enabled to perform health care screening for the wearer of the HMD as discussed herein. Prior to performing the health care screening for a wearer, at step 412 the wearer of the HMD provides opt-in consent to the process 400. In some embodiments, the wearer may provide the opt-in may directly via the HMD. In other embodiments, the wearer may use a network-connected device such as a phone or computer to provide the opt-in. As part of step 412, the wearer's opt-in consent is communicated to the analysis agent. The opt-in consent is the indication that the wearer consents to allowing images of their eyes and ocular facial region to be generated while they are wearing the HMD, along with transmission of those generated images to the analysis agent. If a wearer elects not to provide opt-in consent, then the process 400 terminates.

Following receipt of the opt-in consent, at step 414 contextual training images of the wearer are received from the HMD. At step 416, the training images of the wearer are used to further train the detection model. The training images provide contextual training for the detection model using images of the eyes and ocular facial region of the wearer while the head-mounted is being worn. This contextual training helps the detection model to account for differences between the environment in which the pre-training images were generated (e.g., a medical office or images of a wearer's ocular facial region generated under different environmental circumstances) and environment in which health care screening images of the wearer are taken (e.g., the space between the wearer's face and the display screen of the HMD. Such differences may include brightness, coloration, and positioning of the light source, camera capabilities, positioning of the camera with respect to the eyes, the skin color and skin tone of the individual (whether the patients used to generate the pre-training images or the wearer of the HMD), and any skin products and/or makeup worn by the individual.

Only the training images received from the HMD are processed through the contextual training stage 404. In some embodiments, the contextual training images may be generated using a training process that is performed when the wearer first provides the opt-in consent. In such an embodiment, all contextual training images may be obtained during a single session of the training process. In some embodiments, the training process, and generation of the contextual training images, may be performed over several sessions of training process until such time as sufficient contextual training images are generated as per the predetermined parameters of the training process. Once the contextual training stage 404 has processed the contextual training images, all other images generated by and received from the HMD are processed through the health care screening stage 406. At step 418, the analysis agent receives screening images of the wearer from the HMD. At step 420, the analysis agent analyzes the screening images using the pre-trained and contextually trained neural network. From the analysis, at step 422 the analysis agent determines if symptoms are detected in the screening images. If no symptoms are detected, at step 424, the analysis agent may send additional imaging instructions to the HMD, and such additional imaging instructions may include a notice to the wearer that no symptoms have been detected. In some embodiments, the HMD may present this notice to the wearer on the display screen or via any other user device in communication with the HMD. In some embodiments, the imaging instructions do not include any notice to the wearer when no symptoms are detected. When symptoms are detected, at step 426 the analysis agent sends imaging instructions to the wearer with a notice that symptoms have been detected and indicating the condition or disease that has been potentially identified as part of the health care screening process. The HMD may present this notice of a positive screening result to the wearer on the display screen or via any other user device in communication with the HMD. In some embodiments, notice the wearer may be delivered via email or any other available communications method chosen by the wearer. At step 428, if the option to notify a health care provider is selected and authorized by the wearer, the analysis agent may provide the wearer's selected health care provider with the notice that symptoms have been detected. In addition, the analysis agent may forward the screening images in which the symptoms were identified to the chosen health care provider. In some embodiments, the HMD may forward the notice that symptoms were detected and the screening images to the wearers chosen health care provider. When symptoms have been identified in screening images, those screening images are also directed back to the contextual training stage 404, where at step 416 those screening images are used to reinforce training for the neural network model. In some embodiments, in which past screening images are stored and available, the past screening images that resulted in a negative health screening determination (i.e., no diseases and/or conditions identified) may be used for further training once a positive health screening determination (i.e., at least one disease and/or condition identified) has been made. In such instances, the past screening images may provide additional training to the neural network model that aids in the earlier detection of the same symptoms in future screening images, thereby improving the accuracy of detection.

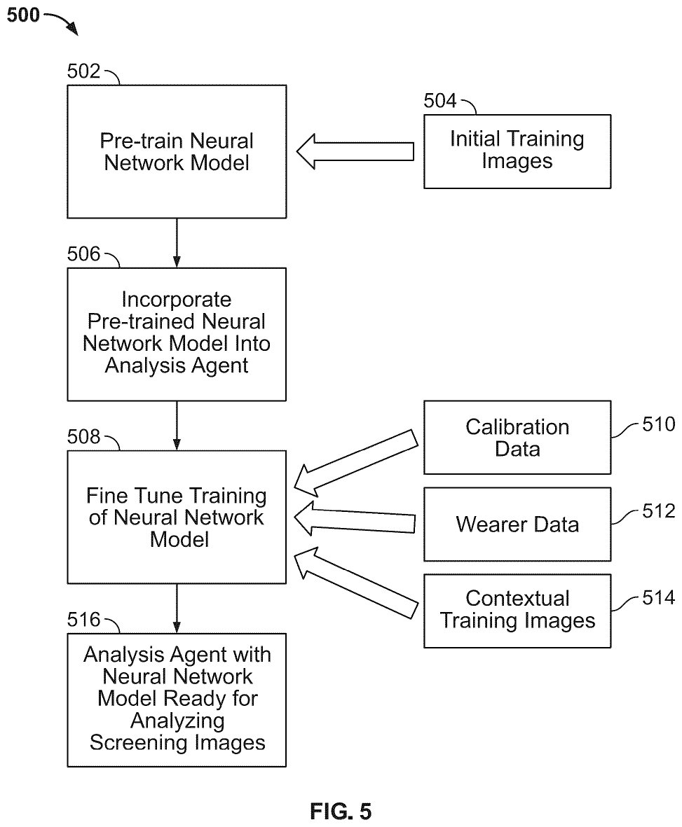

FIG. 5 shows a flowchart illustrating the steps of a process 500 for enabling training of the neural network model, thereby enabling the analysis agent to interact with an HMD to perform health care screening. The process 500 may be implemented on the analysis agent 104 in support of the health care screening process 300. One or more actions of the process 500 may be incorporated into or combined with one or more actions of any other process or embodiments described herein. The process 500 begins at step 502 with pre-training the neural network model. As discussed above, initial training images 504 are used as the input for this pre-training step 502. The initial training images are images of symptomatic patients, and they are typically generated in a medical office. In some embodiments, a subset or all of the training images may be generated in other environments outside of a medical office. At step 506, the pre-trained neural network model is incorporated into an analysis agent. As part of the analysis agent, the neural network model serves as a baseline for analyzing images generated by and received from an HMD. In some embodiments, an analysis agent may use a single pre-trained neural network model to service more than one head-mounted display. In other embodiments, an analysis agent and the associated pre-trained neural network model may be dedicated to a single head-mounted display.

At step 508, the training of the neural network model is fine-tuned using data received from the HMD. The data received for fine tuning the neural network may include calibration data 510, wearer data 512, and contextual training images 514. The calibration data 510 includes information about the HMD and its operational parameters. Such information about the HMD informs the analysis agent about the hardware (e.g. specifications of the imaging subsystem, including the camera(s), display specifications the availability of audio output to the wearer, and the like) associated with the HMD. The calibration data can aid the analysis agent in the analysis of images received from the HMD. The wearer data 512 includes the opt-in consent of the wearer. In some embodiments, the wearer data 512 may also include the age and/or gender of the wearer, ethnicity of the wearer, and any previous diagnoses the wearer may have with respect to the disease and/or conditions for which the health care screening process is screening. In some embodiments, the previous diagnoses may be provided directly by the wearer. In addition, in some embodiments, the head-mounted display may access the wearer's existing medical records and/or profile to obtain previous diagnoses of the wearer. The contextual training data 514 includes images of the wearer while wearing the HMD. In some embodiments, the contextual training images 514 may include images of the wearer while the display screen is controlled to generate different lighting conditions. These personalized images aid the analysis agent in the detection of skin color under different lighting conditions within the HMD (which may include infiltration of surrounding light due to the fit of the HMD to the wearer's face). The different lighting conditions may include different colors, hues, saturations, and the like, as the symptoms for different diseases and/or conditions may appear more prominent when subjected to particular lighting conditions. In addition, the personalized images may be generated while the display screen illuminates the eyes an ocular facial region of the wearer with light of a known wavelength and brightness. By controlling the illumination while generating the personalized images, the neural network model is better able to account for differences in screening images when the lighting isn't controlled during regular use of the HMD.

At step 516, the analysis agent with the neural network model is ready to receive screening images for analysis. In some embodiments, when the screening images are determined to have a positive analysis for a symptom and/or condition, the screening image may be used for reinforcement training of the neural network model.

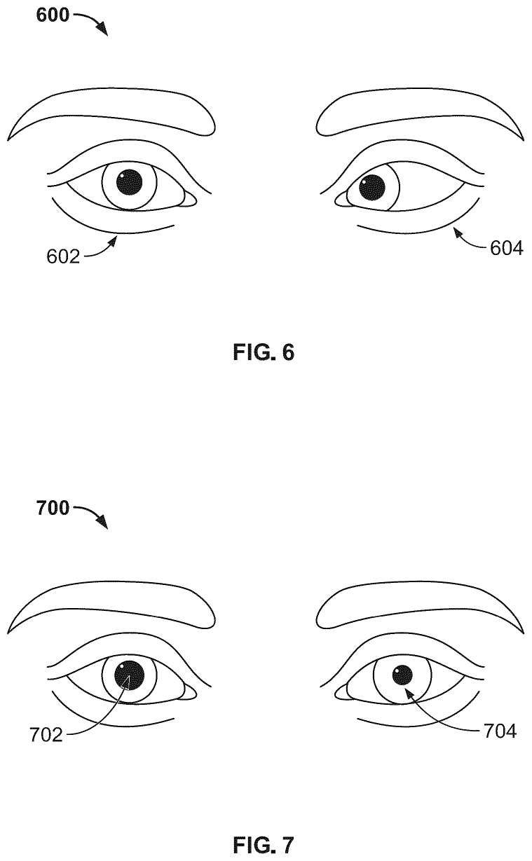

FIGS. 6-9 illustrate how the wearer's eyes may appear in screening images when the health care screening process discussed herein detects symptoms for certain diseases and/or conditions. FIG. 6 illustrates the positioning of a wearer's eyes 600 when the wearer might suffer from the condition strabismus, also referred to as being cross-eyed. This condition is characterized by a misalignment of the eyes, which causes one eye 602 to deviate inward or outward (exotropia) while the other eye 604 remains focused. This condition may be detected by identifying the reflection from the pupillary area of the wearer's eyes and determining that the reflected light is different from each eye. FIG. 7 illustrates a wearer's eyes 700 and the difference between the wearer's pupils 702, 704 when the wearer might suffer from the condition anisocoria. This condition may be characterized by the pupil 702 of one eye being more dilated than the pupil 704 of the other eye. The sizes of their pupils as shown in prior screening images, or even as shown in the contextual training images, may assist the analysis agent in determining which pupil has the typical dilation under conditions of lighting within the HMD and which pupil is enlarged. In some embodiments, the anisocoria condition may be detected using semantic segmentation of the pupils, thereby facilitating evaluation of the area of each pupil.

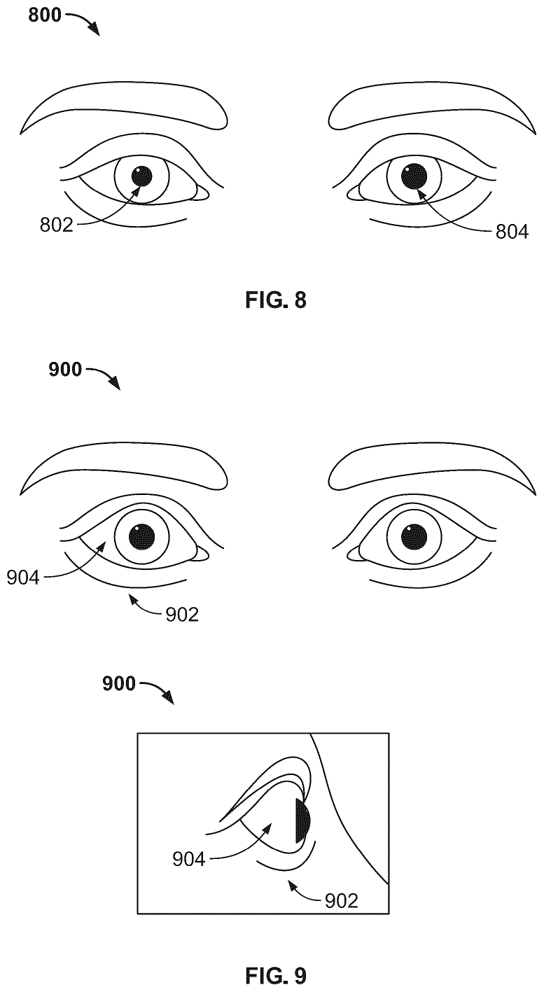

FIG. 8 illustrates a wearer's eyes 800 and the difference between the wearer's pupils 802, 804 when the wearer might suffer from the condition of cluster headaches. This condition may be characterized by the pupil 802 of one eye being more constricted than the pupil 804 of the other eye. The sizes of their pupils as shown in prior screening images, or even as shown in the contextual training images, may assist the analysis agent in determining which pupil has the typical size under conditions of lighting within the HMD and which pupil is more constricted. In some embodiments, the cluster headaches condition may be detected using semantic segmentation of the pupils, thereby facilitating evaluation of the area of each pupil. FIG. 9 illustrates a wearer's eyes 900 as they may appear when the wearer has thyroid disease. This condition may be characterized by the orb 904 of each eye 902 appearing to be bulging and the eyelid 906 of each eye 902 appearing retracted. In embodiments of the HMD having a single camera directed towards each of the wearer's eyes and face, thyroid condition may be most easily detected by the retracted eyelids and additional sclera showing between the eyelids and the irises of the wearer. In embodiments of the HMD having more than one camera directed towards each of the wearer's eyes and face, thyroid condition may also by detected by the bulging of the eyes using images of the eyes taken from multiple angles to identify the bulge in the eyes.

Another disease that may be detectable using the HMD and the health care screening process is diabetic retinopathy. This disease may be characterized by unusual redness of the sclera accompanied by a discharge that may cause the sclera to appear yellowed. Yet another condition that may be detectable using the HMD and the health care screening process is arcus senilis, which in younger individuals (generally under 50 years old) can be a sign of high cholesterol and is characterized by grey, white, or yellow circles around the iris caused by lipid deposits. High cholesterol may also be identified through the screening process by the presence of xanthelasma, which is small, yellowish fatty deposits in the skin around the eyes, particularly in the eyelids.

For certain diseases and/or conditions, the accuracy of the health care screening process may be dependent on the nature of lighting from the display screen. When certain diseases and/or conditions are suspected, the display screen may be used to adjust the lighting to improve the accuracy of the health care screening process. For example, if a certain condition is detected with accuracy of 75% with default lighting from the display screen, and offline experiments have shown that the detection accuracy for this condition can be improved by changing lighting conditions (or, e.g., the pattern of lighting), then the HMD may be instructed to change the lighting conditions to those more favorable for this specific condition while generating at least some of the screening images.

Different diseases and/or conditions may require different lighting conditions to be detected accurately. For example, yellow pigmentation on the skin may be observed in certain lighting conditions, but not in others. Creating the right condition in the HMD may be achieved by appropriate control of the color, brightness, and hue of the light emitted by the display screen. As another example, the brightness of the light from the display screen can be controlled to generate screening images at different dilations. Also, as discussed above, object images may be shown on the display screen, either in a stationary position or moving across the display screen, in order to encourage the wearer to focus their eyes in a desired direction for purposes of generating screening images. As another example, the HMD may be instructed to actively monitor the lighting conditions of the display screen while the HMD is worn, and when the lighting conditions fall within certain predetermined parameters, the HMD generates the screening images.

Certain diseases and/or conditions may be better detected in high contrast images, for example, as compared to images with regular contrast. As such, in some embodiments the screening images may be post-processed to prior to being analyzed by the analysis agent. Such post-processing may be performed by the HMD, the analysis agent, or even by an intermediary device such as a smart phone, laptop computer, or other computing device. In some embodiments, where many contemporaneous screening images are generated by the HMD, some of those images may be post-processed before being analyzed by the analysis agent, and some may not undergo any post-processing. In this manner, the screening images may be useful to detecting a wider range of diseases and/or conditions during the health care screening process.

Certain diseases and/or conditions may be better detected by the combination of multiple images taken under different lighting conditions. For example, one screening image may be taken using visible light, while another is taken using infrared light, and those two images may be overlaid to improve the ability to detect certain diseases and/or conditions in the resulting combined image.

In some embodiments, when multiple related people are using an HMD for health care screening purposes as described herein, and both provide consent, the health care screening results of one may be used to improve the health care screening results of the other. This may be done to improve detection of a disease and/or condition that is known to have a genetic component, where that disease and/or condition has already been detected in one of the related individuals.

FIG. 10 depicts a block diagram of a system 1000 into which an HMD may be incorporated, in accordance with some embodiments of this disclosure. The system is shown to include the HMD 1002, an analysis agent 1004, and a communication network 1006. It is understood that while a single instance of a component may be shown and described relative to FIG. 10, additional embodiments of the component may be employed. For example, the analysis agent 1004 may include, or may be incorporated in, more than one server. Similarly, the communication network 1006 may include, or may be incorporated in, more than one communication network. The analysis agent 1004 is shown communicatively coupled to the HMD 1002 through the communication network 1006. While not shown in FIG. 10, the analysis agent 1004 may be directly communicatively coupled to the HMD 1002, for example, in a system absent or bypassing the communication network 1006.

The communication network 1006 may include one or more network systems, such as, without limitation, the Internet, LAN, Wi-Fi, wireless, or other network systems. In some embodiments, the analysis agent 1004 may work in conjunction with one or more components of the communication network 1006 to implement certain functionality described herein in a distributed or cooperative manner. In some embodiments, the HMD 1002 may work in conjunction with one or more components of the communication network 1006 or the analysis agent 1004 to implement certain functionality described herein in a distributed or cooperative manner.

The HMD 1002 includes control circuitry 1008, a memory 1010, a communications interface 1012, a display screen 1014, an imaging subsystem 1016, and input/output (I/O) circuitry 1018. As shown, the imaging subsystem 1014 includes two cameras 1020, 1022. These cameras 1020, 1022 may be the same cameras used by the HMD when the wearer is using the HMD for other purposes, such as gaming or participating in an XR environment. In some embodiments, the imaging subsystem 1014 may include fewer or more cameras. The control circuitry 1008 may be based on any suitable control circuitry and includes control circuits and memory circuits, which may be disposed on a single integrated circuit or may be discrete components. As referred to herein, control circuitry should be understood to mean circuitry based on at least one of microprocessors, microcontrollers, digital signal processors, programmable logic devices, field-programmable gate arrays (FPGAs), or application-specific integrated circuits (ASICs), etc., and may include a multi-core processor (e.g., dual-core, quad-core, hexa-core, or any suitable number of cores). In some embodiments, the control circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, the control circuitry may be implemented in hardware, firmware, or software, or a combination thereof.

The memory 1010 may be an electronic storage device. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 8D disc recorders, digital video recorders (DVRs, sometimes called personal video recorders, or PVRs), solid state devices, quantum storage devices, gaming consoles, gaming media, or any other suitable fixed or removable storage devices, and/or any combination of the same. The memory 1010 may be used to store several types of content, metadata, and/or other types of data. Non-volatile memory may also be used (e.g., to launch a boot-up routine and other instructions). Cloud-based electronic storage may be used to supplement the memory 1010. In some embodiments, an HMD wearer profile and messages corresponding to a chain of communication may be stored in the memory 1010.

The communications interface 1012 may include any type of communications circuitry that enables the HMD 1002 to communicate, either directly or indirectly, with other devices, servers, networks, and the like. Direct communications circuitry may include those that use protocols such as USB, Bluetooth, serial, and the like. Indirect communications circuitry may include those that use a network (e.g., a local area network (LAN) such as WiFi, a wide area network (WAN) such as the Internet, and the like) interposed between devices. In some embodiments, the communications interface 1012 may be configured for communications using multiple different circuitry and protocols (e.g., Bluetooth, WiFi, 5G, 4G, LTE, Ethernet, USB, etc.). In some embodiments, the communications interface 1012 may include multiple modules, with each module configured for communications using different circuitry and protocol.

The display screen 1014 may be any type of display screen that is suitable for positioning close to the eyes of a wearer of the HMD 1002 and for displaying XR content, multimedia content, and images to the wearer. Such display screens may include LED screens, OLED screens, and the like.

As indicated above, the imaging subsystem 1016 is shown as having two cameras 1020, 1022. Each camera 1020, 1022 is a digital optical camera configured to generate images of the wearer's eyes and optical facial region in the visible spectrum of light. In some embodiments, the cameras 1020, 1022 may include a charge-coupled device (CCD) and/or a complementary metal-oxide semiconductor (CMOS) type image sensor. In some embodiments, more or fewer optical cameras may be included as part of the imaging subsystem 1016. In those embodiments in which multiple cameras generate screening images of each eye, it may be desirable to have the cameras image each eye from different angles, as doing so may enhance the ability of the analysis agent to detect symptoms of certain diseases and/or conditions. In some embodiments, the cameras 1020, 1022 may include the capability of capturing video at 8 fps or greater. In some embodiments, the cameras 1020, 1022 may also include the capabilities to generate images in the infrared spectrum of light. In other embodiments, the imaging subsystem 1016 may include additional infrared cameras for this purpose. In some embodiments, the imaging subsystem 1016 may include eye tracking cameras along with software to enable the HMD to track the gaze direction of the wearer's eyes. As such, the camera may be configured to automatically capture images, e.g., in response to detecting a predefined criteria such as the user is looking upwards without tilting his head (eye roll), looking sideways, detecting a sudden change in the size of the eye (e.g., occurs in response to a reaction, fear, etc.).

The I/O circuitry 1018 enables the control circuitry 1008 to receive inputs from the wearer and to provide additional outputs to the wearer. For example, the I/O circuitry 1018 may be configured to enable the control circuitry 1008 to communicate with one or more of a keyboard, speakers, headphones, a microphone, game controller, VR gloves, remote-control device, trackpad, or any other suitable HMD wearer movement-sensitive, audio-sensitive or capture devices, each of which may be used to provide outputs to the wearer or to receive inputs from the wearer. In some embodiments, the control circuitry 1018 may update the display screen 1014 in response to inputs received via the I/O circuitry 1018, such as while the wearer of the HMD 1002 is playing a video game or exploring an XR environment. In some embodiments, the I/O circuitry 1018 may be configured to provide a wired connection, such as an audio cable, USB cable, ethernet cable and the like attached to a corresponding input port at a local device. In some embodiments, the I/O circuitry 1018 may be configured to provide a wireless connection, such as Bluetooth, Wi-Fi, WiMAX, GSM, UTMS, CDMA, TDMA, 8G, 4G, 4G LTE, 5G, or any other suitable wireless transmission protocol. As such, the I/O circuitry 1018 may include a physical port such as an audio jack, USB port, ethernet port, or any other suitable connection for receiving input over a wired connection and/or the I/O circuitry 1018 may include a wireless transceiver configured to send and receive data via Bluetooth, Wi-Fi, WiMAX, GSM, UTMS, CDMA, TDMA, 3G, 4G, 4G LTE, 5G, or other wireless transmission protocols.

In some embodiments, the control circuitry 1008 executes instructions for an application stored in the memory 1010. Specifically, the control circuitry 1008 may be instructed by the application to perform the functions discussed herein. In some embodiments, any action performed by the control circuitry 1008 may be based on instructions received from the application. For example, the application may be implemented as software or a set of and/or one or more executable instructions that may be stored in the memory 1010 and executed by the control circuitry 1008. The application may be a client/server application where only a client application resides on head-mounted display 1002, and a server application resides on the analysis agent 1004, such that the client application and the server application provide instructions to have the HMD 1002 and the analysis agent 1004 cooperatively perform the functions discussed herein.

The application may be implemented using any suitable architecture. For example, it may be a stand-alone application wholly implemented on the HMD 1002. In such an approach, instructions for the application are stored locally (e.g., in the memory 1010), and data for use by the application is downloaded on a periodic basis (e.g., from an out-of-band feed, from an Internet resource, or using another suitable approach). The control circuitry 1008 may retrieve instructions for the application from the memory 1010 and process the instructions to perform the functionality described herein. Based on the processed instructions, the control circuitry 1008 may determine a type of action to perform in response to input received from the I/O circuitry 1018, from the server 1002, or from the communication network 1006.

In some embodiments, the HMD 1002 may display media content stored in the memory 1010 while performing the health care screening processes described herein. In some embodiments, the HMD 1002 may access media content, for display to the wearer, through another device, such as a media content server, via the communication network 1006.

Similar to the HMD 1002, the analysis agent 1004 includes control circuitry 1030, a memory 1032, and a communications interface 1034. The control circuitry 1030 may be based on any suitable control circuitry and includes control circuits and memory circuits, which may be disposed on a single integrated circuit or may be discrete components. As referred to herein, control circuitry should be understood to mean circuitry based on at least one of microprocessors, microcontrollers, digital signal processors, programmable logic devices, field-programmable gate arrays (FPGAs), or application-specific integrated circuits (ASICs), etc., and may include a multi-core processor (e.g., dual-core, quad-core, hexa-core, or any suitable number of cores). In some embodiments, the control circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, the control circuitry may be implemented in hardware, firmware, or software, or a combination thereof.

The memory 1032 may be an electronic storage device. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 8D disc recorders, digital video recorders (DVRs, sometimes called personal video recorders, or PVRs), solid state devices, quantum storage devices, gaming consoles, gaming media, or any other suitable fixed or removable storage devices, and/or any combination of the same. The memory 1032 may be used to store several types of content, metadata, and/or other types of data. Non-volatile memory may also be used (e.g., to launch a boot-up routine and other instructions). Cloud-based electronic storage may be used to supplement the memory 1032. In the processes described above, the memory 1032 is used to store data associated with the neural network model, images received from the HMD 1002 for processing, and other data associated with the health care screening process discussed herein. In embodiments where the stored data includes information that may be used to identify the wearer of the HMD 1002, it may be desirable to store the data in an encrypted format.

The communications interface 1034 may include any type of communications circuitry that enables the analysis agent 1004 to communicate, either directly or indirectly, with other devices, servers, networks, and the like, including the HMD 1002. Direct communications circuitry may include those that use protocols such as USB, Bluetooth, serial, and the like. Indirect communications circuitry may include those that use a network (e.g., a local area network (LAN) such as WiFi, a wide area network (WAN) such as the Internet, and the like) interposed between devices. In some embodiments, the communications interface 1012 may be configured for communications using multiple different circuitry and protocols (e.g., Bluetooth, WiFi, 5G, 4G, LTE, Ethernet, USB, etc.). In some embodiments, the communications interface 1034 may include multiple modules, with each module configured for communications using different circuitry and protocol.

The processes described above are intended to be illustrative and not limiting. One skilled in the art would appreciate that the steps of the processes discussed herein may be omitted, modified, combined, and/or rearranged, and any additional steps may be performed without departing from the scope of the invention. More generally, the above disclosure is meant to be illustrative and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

1. A method comprising:
receiving, via a communications interface of a head-mounted display, first imaging instructions from an analysis agent, the first imaging instructions comprising first imaging parameters;
generating, using an imaging subsystem and a display screen of the head-mounted display, first screening images based on the first imaging parameters, the first screening images being of an ocular facial region of a wearer of the head-mounted display;
transmitting, via the communications interface, the first screening images to the analysis agent; and
receiving, via the communications interface, second imaging instructions from the analysis agent.

2. The method of claim 1, wherein generating the first screening images comprises:
displaying, on the display screen, a predetermined scene based on the first imaging parameters; and
generating the first screening images while displaying the predetermined scene to the wearer.

3. The method of claim 2, wherein the predetermined scene comprises one of a video, a still image, and an illumination setting.

4. The method of claim 1, further comprising transmitting, via the communications interface, the first screening images to a network storage.

5. The method of claim 1, wherein the first imaging instructions comprise a schedule for generating first screening images.

6. The method of claim 1, wherein the first imaging instructions comprise a preliminary screening analysis based on the first screening images.

7. The method of claim 6, further comprising transmitting, via the communications interface, the preliminary screening analysis to a health care system.

8. The method of claim 1, wherein second imaging instructions comprise second imaging parameters.

9. The method of claim 8, further comprising:
generating, using the imaging subsystem and the display screen, the second screening images based on the second imaging parameters, the second screening images being of the ocular facial region of the wearer of the head-mounted display;
transmitting, via the communications interface, the second screening images to the analysis agent; and
receiving, via the communications interface, third imaging instructions from the analysis agent.

10. The method of claim 1, wherein generating the first screening images comprises:
displaying, on the display screen, an object image based on the first imaging parameters; and
generating the first screening images while eyes of the wearer track to the object image.

11. The method of claim 10, wherein displaying the object image comprises displaying the object image moving on the display screen.

12. The method of claim 1, wherein the imaging subsystem comprises at least one camera.

13. A head-mounted display system comprising:
a communications interface;
an imaging subsystem;
a display screen;
control circuitry configured to:
receive, via the communications interface, first imaging instructions from the analysis agent, the first imaging instructions comprising first imaging parameters;
generate, using the imaging subsystem and the display screen, the first screening images based on the first imaging parameters, the first screening images being of an ocular facial region of a wearer of the head-mounted display;
transmit, via the communications interface, the first screening images to the analysis agent; and
receive, via the communications interface, second imaging instructions from the analysis agent.

14. The system of claim 13, wherein, to generate the first screening images, the control circuitry is further configured to:
display, on the display screen, a predetermined scene based on the first imaging parameters; and
generate the first screening images while displaying the predetermined scene to the wearer.

15. The system of claim 14, wherein the predetermined scene comprises one of a video, a still image, and an illumination setting.

16. The system of claim 13, wherein the control circuitry is further configured to transmit, via the communications interface, the first screening images to a network storage.

17. The system of claim 13, wherein the first imaging instructions comprise a schedule for generating the first screening images.

18. The system of claim 13, wherein the second imaging instructions comprise a preliminary screening analysis based on the first screening images.

19. The system of claim 18, wherein the control circuitry is further configured to transmit, via the communications interface, the preliminary screening analysis to a health care system.

20. The system of claim 13, wherein the second imaging instructions comprise second imaging parameters.

21-50. (canceled)
